SEQUENCE LISTING (6 sequences)

SEQ ID NO: 1 - RNA, 23 nt, E. Coli cat Gene
cggugguauauccagugauuuuu

SEQ ID NO: 2 - DNA, 23 bp, E. Coli cat Gene
aaatcactggatataccaccgtt

SEQ ID NO: 3 - RNA, 23 nt, E. Coli cat Gene
cuggccagccacauaggaguuuu

SEQ ID NO: 4 - DNA, 23 bp, E. Coli cat Gene
aactcctatgtggctggccagtt

SEQ ID NO: 5 - RNA, 23 nt, E. Coli folA Gene
ucucgccugguuuaaacgcaauu

SEQ ID NO: 6 - DNA, 23 bp, E. Coli folA Gene
ttgcgtttaaaccaggcgagatt

(19) United States
(12) Patent Application Publication
(10) Pub. No.: US 2004/0053289 A1
(43) Pub. Date: Mar. 18, 2004

(54) Short interfering nucleic acid hybrids and methods thereof

(75) Inventors: Allen T. Christian, Tracy, CA (US); Janelle S. Lamberton, Livermore, CA (US)

Correspondence Address: John H. Lee, Assistant Laboratory Counsel, Lawrence Livermore National Laboratory, P.O. Box 808, L-703, Livermore, CA, 94551, US

(73) Assignee: The Regents of the University of California

(21) Appl. No.: 10/410,220

(22) Filed: Apr. 8, 2003

Related U.S. Application Data
(60) Provisional application No. 60/409,680, filed on Sep. 9, 2002.

(51) Int. Cl. C12Q 1/68; C07H 21/04

(52) U.S. Cl. 435/6; 536/24.3

(57) ABSTRACT

Disclosed herein are siHybrids used for gene silencing. An siHybrid is a short double-stranded molecule comprised of one strand of DNA and one strand of RNA, annealed together, with a 2-base overhang at each 3′ end. In addition to DNA and RNA, it may contain PNA or other nucleic acid analogs. siHybrids can silence a gene with greater magnitude and duration than siRNA and they can also silence bacterial genes, which siRNA cannot. siHybrids are ideal candidates for pharmaceutical and therapeutic agents for treating diseases caused by an over-expressed gene or a cancerous gene. They also can be used as antibiotics when targeted to a vital and unique bacterial gene. siHybrids can be used as antivirus agents, fungicides, herbicides or pesticides. An appropriate siHybrid can be designed to silence any gene in any cell of any organism.

### CROSS-REFERENCE TO RELATED APPLICATIONS

This application claims the benefit of U.S. Provisional Application No. 60/409,680, filed Sep. 9, 2002, titled “Gene Silencing Using DNA:RNA-Hybrid Short, Interfering Molecules” and is incorporated herein by this reference.

The United States Government has rights in this invention pursuant to Contract No. W-7405-ENG-48 between the United States Department of Energy and the University of California for the operation of Lawrence Livermore National Laboratory.

### BACKGROUND

In recent years it has been accepted that RNA interference is mediated by short interfering RNA molecules (“siRNA”) that exhibit sequence specific gene silencing effects. Although the detailed mechanism of siRNA gene silencing is not fully understood, genes can be silenced or disabled by degradation of cellular mRNA by introducing an siRNA molecule that is homologous to the target genes.

Previous experimental work involving the use of antisense molecules demonstrated antisense therapy as an excellent antiviral infectant, but its utility was offset by the fact that the half-life of antisense molecules is very short. Also, antisense therapy is a passive process in that it simply blocks the translation of the viral mRNA, whereas RNAi actually degrades the mRNA. Similar work involving the transfection of an siRNA-producing plasmid into cells works well for mutagenesis studies, but an active process such as this may not be as useful for long-term protection from a genetic process, such as microbial infection.

The following references are related to gene silencing technology and are hereby incorporated by reference in their entirety.

References:

1. Fire, A., Xu, S., Montgomery, M. K., Kostas, S. A., Driver, S. E., and Mello, C. C. (1998) Potent and specific genetic interference by double stranded RNA in Caenorhabditis elegans. Nature. 408, 325-330.

2. Kennerdell, J. R., and Carthew, R. W. (1998) Use of dsRNA-mediated genetic interference to demonstrate that frizzled and frizzled 2 act in the wingless pathway. Cell. 95, 1017-1026.

3. Misquitta, L., and Paterson, B. M. (1999) Targeted disruption of gene function in Drosophila by RNA interference (RNA-i): a role for nautilus in embryonic somatic muscle formation. Proc. Natl. Acad. Sci. USA. 96, 1451-1456.

4. Hammond, S. M., Bernstein, E., Beach, D., and Hannon, G. J. (2000) An RNA-directed nuclease mediates post transcriptional gene silencing in Drosophila cells. Nature. 404, 293-296.

5. Lohmann, J. U., Endl, I., and Bosch, T. C. (1999) Silencing of developmental genes in Hydra. Dev. Biol. 214, 211-214.

6. Wargelius, A., Ellingsen, S., and Fjose, A. (1999) Double stranded RNA induces specific developmental defects in zebrafish embyos. Biochem. Biophys. Res. Commun. 263, 156-161.

7. Ngo, H., Tschudi, C., Gull, K., and Ullu, E. (1998) Double stranded RNA induces mRNA degradation in Trypanosoma brucei. Proc. Natl. Acad. Sci. USA. 95, 14687-14692.

8. Montgomery, M. K., Xu, S., Fire, A. (1998) RNA as a target of double stranded RNA mediated genetic interference in Caenorhabiditis elegans. Proc. Natl. Acad. Sci. USA. 95, 15502-15507.

9. Bosher, J. M., Dufourcq, P., Sookhareea, S., Labouesse, M. (1999) RNA interference can target pre-mRNA. Consequences for gene expression in Caenorhabiditis elegans operon. Genetics. 153, 1245-1256.

10. Fire, A. (1999) RNA-triggered gene silencing. Trends Genet. 15, 358-363.

11. Sharp, P. A. (1999) RNAi and double-stranded RNA. Genes Dev. 13, 139-141.

12. Ketting, R. F., Harerkamp, T. H., van Luenen, H. G., and Plasterk, R. H. (1999) Mut-7 of C. elegans, required for transposon silencing and RNA interference, is a homolog of Werner syndrome helicase and RNase I. Cell. 99, 133-141.

13. Tabara, H., Sarkissian, M., Kelly, W. G., Fleenor, J., Grishok, A., Timmons, L., Fire, A., and Mello, C. C. (1999) The rde-1 gene, RNA interference, and transposon silencing in C.elegans. Cell. 99, 123-132.

14. Zamore, P. D., Tuschl, T., Sharp, P. A., and Bartel, D. P. (2000) RNAi: Double stranded RNA directs the ATP dependent cleavage of mRNA at 21 to 23 nucleotide intervals. Cell. 101, 25-33.

15. Bernstein, E., Caudy, A. A., Hammond, S. M., and Hannon, G. J. (2001) Role for a bidentate ribonuclease in the initiation step of RNA interference. Nature. 409, 363-366.

16. Elbashir, S., Lendeckel, W., and Tuschl, T. (2001) RNA interference is mediated by 21 and 22 nucleotide RNAs. Genes and Dev. 15, 188-200.

17. Sharp, P. A. (2001) RNA interference 2001. Genes and Dev. 15, 485-490.

18. Hunter, T., Hunt, T., and Jackson, R. J. (1975) The characteristics of inhibition of protein synthesis by double-stranded ribonucleic acid in reticulocyte lysates. J. Biol. Chem. 250, 409-417.

19. Bass, B. L. (2001) The short answer. Nature. 411, 428-429.

20. Elbashir, S. M., Harborth, J., Lendeckel, W., Yalcin, A., Weber, K., and Tuschl, T. (2001) Duplexes of 21-nucleotide RNAs mediate RNA interference in cultured mammalian cells. Nature. 411, 494-498.

21. Carson, P. E. and Frischer, H. (1966) Glucose-6-Phosphate dehydrogenase deficiency and related disorders of the pentose phosphate pathway. Am J Med. 41, 744-764.

22. Stamato, T. D., Mackenzie, L., Pagani, J. M., and Weinstein, R. (1982) Mutagen treatment of single Chinese Hamster Ovary cells produce colonies mosaic for Glucose-6-phosphate dehydrogenase activity. Somatic Cell Genetics. 8, 643-651.

### SUMMARY OF THE INVENTION

The present invention provides a novel composition and method of using the composition to inhibit gene function in any organism or cell, both prokaryotes and eukaryotes in vivo and in vitro. The short interfering nucleic acid or nucleic acid analog hybrids of this invention may be used to target and inhibit the function of any gene for which a specific sequence can be identified regardless of the function or the source of the gene.

In specific embodiments, the present invention provides a composition that is composed of hybridized complimentary portions of single strands of nucleic acids or nucleic acid analogs that are hybridized to other single strands of different types of nucleic acids or nucleic acid analogs to form an siHybrid that has a hybridized portion and at least one 3′ overhang. The hybridized portion of the sihybrid may be as long as from ten to one hundred base pairs in length, depending on the gene and the organism or cell to which it is to be applied.

The present invention also provides a composition that is composed of hybridized complimentary portions of single strands of nucleic acids or nucleic acid analogs that are hybridized to other single strands of different types of nucleic acids or nucleic acid analogs to form an siHybrid that has a hybridized portion that has a length of 19 to 21 base pairs and two 3′ overhangs that are 2-3 bases in length.

Additionally, the present invention provides a composition that is composed of hybridized complimentary portions of single strands of nucleic acids or nucleic acid analogs that are hybridized to other single strands of different types of nucleic acids or nucleic acid analogs to form an siHybrid that has a hybridized portion that has a length of 21 base pairs and two 3′ overhangs that are 2 bases in length.

The invention also provides a method for making the siHybrid compositions by providing single strands of nucleic acids or nucleic acid analogs that are hybridized to other single strands of different types of nucleic acids or nucleic acid analogs to form an sihybrid that has a hybridized portion and at least one 3′ overhang.

The invention furthermore provides a method for making the siHybrid compositions by providing single strands of nucleic acids or nucleic acid analogs that are hybridized to other single strands of different types of nucleic acids or nucleic acid analogs to form an siHybrid that has a hybridized portion that has a length of 19 to 21 base pairs and two 3′ overhangs that are 2-3 bases in length.

Additionally, the invention provides a method for making the siHybrid compositions by providing single strands of nucleic acids or nucleic acid analogs that are hybridized to other single strands of different types of nucleic acids or nucleic acid analogs to form an siHybrid that has a hybridized portion that has a length of 21 base pairs and two 3′ overhangs that are 2 bases in length.

The invention also provides a method for making a plurality of siHybrid compositions by providing multiple single strands of nucleic acids or nucleic acid analogs that are hybridized to other multiple single strands of different types of nucleic acids or nucleic acid analogs to form a plurality of siHybrids that have hybridized portions that have a length of 19 to 21 base pairs and at least one 3′ overhang that is 2 to 3 bases in length.

The invention also provides a method for making a plurality of siHybrid compositions by providing multiple single strands of nucleic acids or nucleic acid analogs that are hybridized to other multiple single strands of different types of nucleic acids or nucleic acid analogs to form a plurality of siHybrids that have hybridized portions that have a length of 21 base pairs and two 3′ overhangs that are 2 bases in length.

A further embodiment of the invention is a method of applying the siHybrids directly to a substrate or to a substrate using a transfecting agent to silence a single gene or a plurality of genes, where the substrate is a cell or an organism that is a virus, a prokaryote, a bacterium, a eukaryote, eukaryotic cells, a vertebrate, a mammal, a primate, a human, human cells, a plant, an insect, or a fungus.

### BRIEF DESCRIPTION OF THE DRAWINGS

FIG. 1 is an illustration of an siHybrid molecule.

FIG. 2 shows gene silencing of G6PD by unaided delivery of siRNA and siHybrid molecules in mammalian cells.

FIG. 3 is a graph showing that the nucleic acid conformation of the short interfering molecules alters the degree and the persistence of the siRNA-mediated gene silencing effects in mammalian cells.

FIG. 4 is a graph showing responses to the degree and length of gene silencing effects in two types of mammilian cells.

FIG. 5 shows CFU formation in bacterial cells silencing an antibiotic resistant gene.

FIG. 6 shows CFU formation in bacterial cells silencing the folA gene.

### DETAILED DESCRIPTION

The three greatest weaknesses of siRNA are its short term effects, its ineffectiveness on bacteria, and the requirement for aided delivery to cells. Transfection is a strategy to deliver genes and other nucleic acids into eukaryotic cells. There are three categories of transfection techniques: biochemical methods, physical methods and virus mediated methods. The transfection technique used is determined by the stress of the transfection on the cells and the efficiency of the method. Biochemical approaches include calcium-phosphate mediated, DEAE-dextran mediated, and lipotransfection. Physical methods include electroporation and biolistics. In bacteria the uptake of nucleic acid is called transformation. The membranes of bacteria must be treated to allow the cells to be “competent” to take up foreign nucleic acid. The two transformation techniques are heat shock and electroporation.

Short duration is a characteristic of siRNA that prevents any meaningful clinical use. Potential applications including cancer therapies, antiviral agents, and cures for certain genetic diseases all require a long-acting process to facilitate delivery and effectiveness. Uses that will accommodate a shorter-lived treatment, as an antibacterial agent, for example, are eliminated due to siRNA's ineffectiveness on bacteria. The siHybrid construction disclosed herein solves both of these problems.

Disclosed herein are siHybrid molecules that have similar function to siRNA, but are much more effective at gene silencing. Instead of a double-stranded RNA molecule, an siHybrid molecule comprises one strand of nucleic acid, e.g., RNA, hybridized to a second strand of nucleic acid that is a different type of nucleic acid than the first strand, e.g., DNA. The siHybrid created by the hybridization of the two different types of nucleic acid have a hybridized complimentary portion and at least one 3′ overhanging end. Nucleic acid analogs can be used in place of nucleic acids. The term “nucleic acid analog” refers to modified or non-naturally occurring nucleotides or backbone structures, such as peptide nucleic acid (PNA).

The unique functions of siHybrids may relate to the stability of the molecule. A double-stranded RNA molecule is inherently unstable; it is rapidly degraded in mammalian cells, and almost instantly degraded in bacteria. A DNA:RNA hybrid, in contrast, is the most stable sort of nucleic acid molecule possible from natural materials, and the construct is not degraded in cells, bacterial or mammalian. Experimental results indicate that the DNA:RNA hybrid is a more potent gene silencing agent than siRNA. Logically, the more stable the molecule is, the more potent a gene silencing agent the molecule can be. Therefore, an siHybrid comprising at least one PNA, or a molecule made of new synthetic nucleic acid analogs, might be equally effective or more potent than a DNA:RNA hybrid, if the synthetic siHybrid is more stable than a DNA:RNA hybrid.

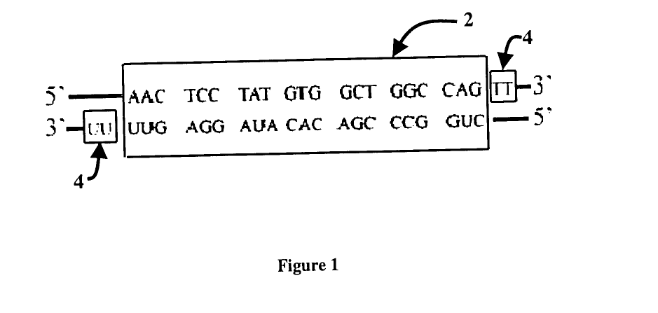

Referring to FIG. 1, the most effective siHybrids have a hybridized complimentary portion (2) that is 19 to 21 base pairs in length and at least one overhanging 3′ end (4) that is at least 2 bases in length. The hybridized complimentary portion of the molecule can be up to 100 base pairs. Generally, the shorter the length is, the less the specificity there will be. If the siHybrid contains less than ten base pairs, it will lose specificity for silencing a gene. On the other hand, a long molecule will have difficulty entering a cell, and therefore cannot silence the gene. Thus, an siHybrid containing more than 100 base pairs will have difficulty entering a cell.

An siHybrid with a sequence common to more than one gene can be used to silence multiple genes simultaneously. Also, multiple siHybrids can be used to silence multiple genes. Multiple gene silencing is especially useful for antibiotic purposes, because by silencing more than one gene simultaneously, it may be able to kill bacteria more selectively and efficiently than by silencing only one gene. Multiple gene silencing is also useful for human therapeutic purposes. For example, by suppressing multiple genes responsible for tumor growth, efficient inhibition of the tumor's growth that may not be achieved by suppressing just one gene can be effected.

siHybrid molecules have near universal potential. They can be used to silence genes in the cell(s) of any organism. They can be used for therapy or research purposes. They can be used as an antibiotic, antiviral agents, and cancer therapy agents and can also be used to treat various genetic diseases caused by the unwanted over-expression of a gene. In addition, they can be used in plants to cure plant diseases, improve plant traits, such as yield, color, environmental tolerance, or quality. By selectively silencing a gene(s), siHybrids can be used as herbicides, insecticides, pesticides and fungicides.

siHybrids can be used to prevent viral infection of cells. By finding the genes that are unique and essential to virus infection, such as proteinase genes or reverse transcriptase genes, constructing corresponding siHybrids and applying those siHybrids to cells, the virus can be killed and viral,infection can be cured by silencing the genes. In addition, an siHybrid can be used as an antibiotic by silencing essential gene pathways of bacterial strains. Silencing such pathways provides a means.of killing the bacterial cells.

Furthermore, siHybrids can be used to treat human or animal diseases resulting from over-expression of genes or disease causing genes. Such diseases may include, but are not limited to, autoimmune diseases, tumors, inflammatory disease and hypertension. siHybrids can also be used to suppress normally expressed genes for therapeutic purposes. For example, to enable successful organ transplants, genes relating to immune response for rejection can be suppressed.

Formulation and Routes of Administration:

siHybrids may be formulated in any pharmaceutically acceptable dosage form. For example, the dosage form may be one suitable for intravenous administration in humans. The dosage forms may include pharmaceutically acceptable excipients, carriers, buffers, osmotic agents and the like, which are known in the art. The formulation may include other pharmaceutically active ingredients for combinational therapies. The formulation may also be designed for a specific utility, in a powder, solid, liquid or gaseous form. siHybrids can be administered orally, subcutaneously, intravenously, intracerebrally, intramuscularly, intramedullary, pareternally, transdermally, nasally or rectally. The form the siHybrids are administered depends at least in part on the route by which they are administered.

Experimental:

Experiments were conducted on mammillian cells and bacteria cells as outlined in the sections below. The concentration of siHybrid used in mammillian cell experiments ranged from 10 μg per 1×10⁶ cells to 25 μg per 1×10⁶ cells in final concentration. The concentration of siHybrid used in bacterial cell experiments ranged from 0.25 μg per milliliter to 4 μg per milliliter in final concentration. Although these experiments demonstrated that the range was effective in silencing genes, the actual lowest effective concentration could be much lower than 10 μg per 1×10⁶ cells or 0.25 μg per milliliter.

Mammalian Cell Summary:

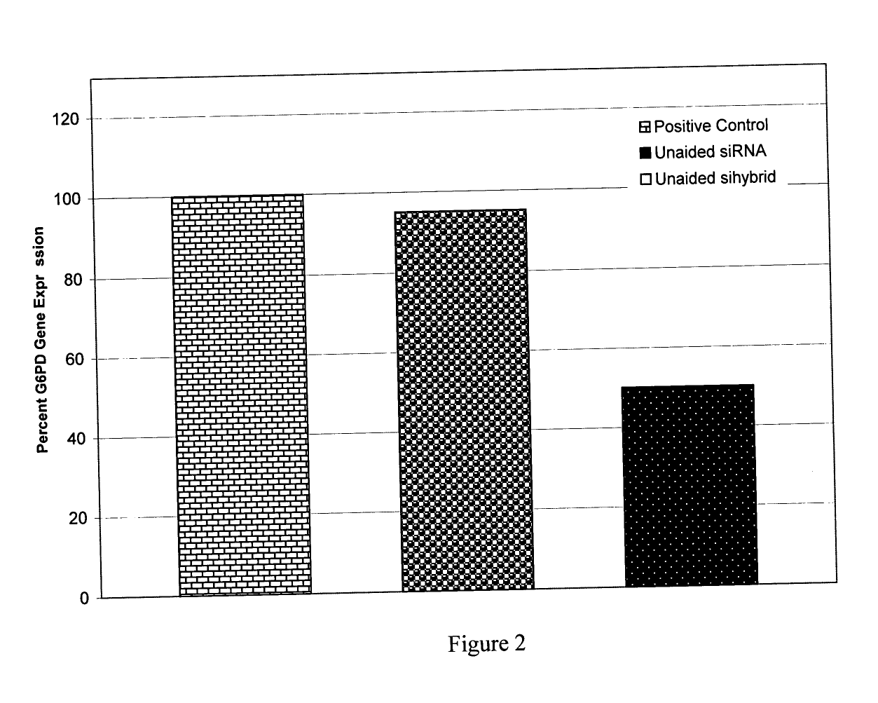

A process was developed to test the effects of siHybrids on various oncogenes and tumor suppressor genes. The goal was to develop a way to shut off a particular gene for a long time, and observe the effects. siHybrids were used to silence the glucose-6-phosphate dehydrogenase (G6PD) gene in normal and cancerous cells of human and hamster origin. The results showed that siHybrids were more potent than siRNA and siDNA in suppressing G6PD gene expression, both in magnitude and duration. The results also showed that the potency of siHybrid is independent of the DNA:RNA orientation. In the siRNA and siHybrid gene silencing experiments only lipotransfection was used. Lipotransfection involves coating the nucleic acid to be delivered into the cells with cationic lipids that bind to the nucleic acid molecules. The artificial membrane fuses with the cell membrane, which is also made of lipids but is negatively charged. For unaided delivery experiments the constructs were added directly to the media. No transfection media or agents were necessary; simply adding the siHybrids to the media was sufficient. FIG. 2 shows gene silencing of G6PD by unaided delivery of siRNA and siHybrid molecules.

In a different experiment, siHybrids were added to dividing cells and then grown for at least eight days. At various intervals during the eight days, attempts were made to induce the G6PD gene, and less than 40% gene expression was observed. Control cells showed normal G6PD activity, and cells in which conventional siRNA molecules had been added showed that normal G6PD activity returned to 100% gene expression within two days. These observations show that siHybrids can be used to silence almost all genes in mammalian cells. This function can be used to suppress any disease causing gene over expression, thus providing an effective treatment for the disease.

Bacterial Cell Summary:

The remarkable efficacy of gene silencing using siHybrids on mammalian cells suggested its use on bacterial cells, under the logical premise that its remarkable longevity in mammalian cells would allow it to act similarly in bacteria. The first targets were simple: antibiotic resistance genes located on plasmids transformed into the bacteria. When grown in media containing antibiotics, only the activity of the genes contained on these plasmids allows the bacteria to survive. E. coli was used for all bacterial tests, since they are readily available and safe to use. As the mechanism by- which bacteria degrade siRNA is purported to be the same in all bacteria, it is assumed that this test case is reasonable. In a series of experiments, genes providing resistance to ampecillin and chloramphenicol, two common antibiotics, were silenced. In all cases, bacteria that were able to grow readily in antibiotic-containing media died when siHybrids against the antibiotic resistance gene were added to the media. No transforming steps, nor any transfection media or agents, were necessary; simply adding the siHybrids to the media was sufficient. Also, when siHybrids not targeted to an expressed gene were added to the media, there was no effect; the bacteria grew exactly as if nothing had been added to them. This demonstrated that it was a directed effect that had been observed, not a wholesale, nonspecific killing of the cells.

To confirm the gene silencing effect in bacteria, the folA gene, which produces the DHFR protein and provides a means for purine synthesis in bacteria, was targeted. In ‘rich’ media, which is supplied with purines, the gene product is unnecessary. When the DHFR silencing siHybrids were added to bacteria growing in rich media, there was no effect. In minimal media, however, which is not supplied with purines or pyrimidines, the DHFR protein is necessary for cell survival. It allows the cells to synthesize purines out of other molecules, such as thymidine or adenine. In minimal media, if the folA gene is not functional, then the cells cannot undergo DNA synthesis, and will thus become quiescent and will not grow. Upon addition of a purine or pyrimidine (nucleoside), such as adenine or thymidine, to the media, the cells will begin to grow again since the DHFR protein is no longer required for purine synthesis. For this experiment, we added the siHybrid folA antagonist to the media, and then added ampecillin. All cells that acquired the siHybrids stopped dividing; those that did not were killed by the ampecillin, which only kills dividing cells. The cells were spun out of the ampecillin-containing media, and resuspended in minimal media containing neither siHybrids, ampicillin nor nucleosides. Immediately upon addition of purines to the media, the cells began growing at a normal rate. Again, adding siHybrids targeted toward non-essential or unexpressed genes had no effect. Adding siRNA molecules of the same sequence as the siHybrids also had no effect. These experiments show that the gene silencing was both specific and non-lethal, and not possible with conventional siRNA treatments. The results described in this experiment show that by selecting a unique gene, necessary for bacterial vitality, for specific bacteria, siHybrids can act as an antibiotic for those specific bacteria by silencing the gene.

Because bacteria have cell walls and membranes, the results of this experiment suggests that siHybrids can be directly applied to mammalian cells to exert gene silencing effects rather than using transfection means because mammalian cells only have a cell membrane that is easier for siHybrids to penetrate and siHybrids are relatively more stable than siRNA. Therefore, in addition to delivering siHybrid to cells via transfection means, such as liposomes, proteins and nucleic acid sequences, siHybrids can also be used directly for disease treatment without transfection agents.

Mammalian Cells

To explore the capabilities of RNAi mediated by siRNA an experiment was designed to post transcriptionally silence an inducible, endogenous gene in cultured mammalian cells, and to determine the duration of this effect. siRNA was used to silence the glucose-6-phosphate dehydrogenase (G6PD) gene in the CHO AA8 cell line, an inducible and endogenous gene found in mammalian cells. G6PD plays an important role in the pentose phosphate pathway in animal tissues to generate the reduced form of nicotinamide dinucleotide triphosphate, NADPH and ribose-5-phosphate that is utilized to generate nucleotides (See Carson, P. E. and Frischer, H. (1966) Glucose-6-Phosphate dehydrogenase deficiency and related disorders of the pentose phosphate pathway. Am J Med. 41, 744-764.). Glucose-6-phosphate enters the pathway and is oxidized by G6PD to generate NADPH and 6-phospho-glucno-8-lactone (See Carson, P. E. and Frischer, H. (1966) Glucose-6-Phosphate dehydrogenase deficiency and related disorders of the pentose phosphate pathway. Am J Med. 41, 744-764.). The oxidative reduction properties of this reaction can be used in combination with a tetrazolium based histochemical stain may be used on cells exposed to Glucose-6-phosphate as a colorimetric assay to quantify the degree of G6PD gene silencing as represented by the level of G6PD enzymatic activity in the cells.

An analysis of siRNA mediated gene silencing with variations in the nucleic acid composition of the short interfering molecules was used to test their effects on the parameters influenced by this mode of gene silencing. These factors include the degree and persistence of the gene silencing effects as well as the amount of recovered gene expression. Using the mammalian G6PD gene these parameters are affected depending on the nucleic acid composition of the short interfering molecules the cells are exposed to. To demonstrate the universality of these findings among mammalian cells a comparison analysis between human and hamster cells was performed.

Materials and Methods

Short interfering molecule preparation. A 21 bp sequence was chosen randomly from the G6PD gene sequence. A second region homologous to a sequence in both the hamster and human G6PD gene was used for the hamster-human comparison studies. Sense and antisense strands were constructed with 2 nucleotide 3′ uridine overhangs at DNA Synthesis Core Facility at Johns Hopkins University. SiDNA sequence contained 2 nucleotide 3′ thymidine overhangs. SiRNA sequence were unpurified, siDNA sequences were RP cartridge purified. Sense and antisense strands were annealed together in equimolar amounts in the presence of 10 mM Tris-HCl (pH 8.0) by denaturing for 5 minutes at 94° C. and reannealed at 53° C. for 3 h and then slowly cooled to room temperature.

Cell Culture and Transfection. Chinese Hamster Ovary (CHO) AA8 cells were propagated in F-12 Nutrient Mixture Ham (Life Technologies, New York) supplemented.with 10% Fetal Bovine Serum (FBS), 1% L-glutamine, and 1% antibiotic-antimycotic at 37° C. Human MCF-7 cells were propagated in DMEM/F-12 (Life Technologies) supplemented with 10% FBS, 1% L-glutamine, 1% penicillin-streptomycin, 1% MEM Non essential amino acid solution, 1% sodium pyruvate, and 2% BME amino acid solution. FBS was inactivated by heating for 30 minutes at 56° C. to eliminate nuclease activity. Cells were passed 3 times per week to maintain exponential growth. Twenty four hours prior to transfection cells were washed 3 times with 1×PBS, trypsinized and plated in 35 mm tissue culture dishes at 1×10⁶ cells/plate in 2 ml growth medium without antibiotics and incubated at 37° C. Transfection of short interfering molecules was performed using Lipofectamine Reagent (Life Technologies, New York) according to manufacturer's protocol for adherent cells using 10 μg of nucleic acid. Cells were incubated with transfection complexes for 5 h. To prevent toxicity of the cells, complexes were aspirated and cells were washed 2 times with complete growth medium and incubated at 37° C. in growth medium with antibiotics until ready to assay for G6PD enzymatic activity.

G6PD Colorimetric Assay and Quantification of Enzymatic Activity. G6PD enzymatic activity was monitored as described by Stamato et al, (See Stamato, T. D., Mackenzie, L., Pagani, J. M., and Weinstein, R. 1982) Mutagen treatment of single Chinese Hamster Ovary cells produce colonies mosaic for Glucose-6-phosphate dehydrogenase activity. Somatic Cell Genetics. 8, 643-651). Briefly, monolayers of cells were washed with 2 ml of 0.14 M NaCl/0.012% Triton-X 100 solution and incubated at 37° C. for 1 h in 2 ml of solution containing 2.5 mg/ml glucose-6-phosphate disodium salt, pH 6.5, 0.17 mg/ml phenazine methosulfate, 0.33 mg/ml nitro blue tetrazolium, 0.14 M NaCl, 0.17 mg/ml NADP and 0.012% Triton-X 100. Cells were fixed for 15 min with 2 ml of 10% acetate buffered formalin, washed and dried with nitrogen. To quantify enzymatic activity, average pixel intensities of cells were obtained to represent the degree of color in the cells which is related to the level of G6PD activity. Cells were observed using brightfield light on Zeiss Axiophot at 20×. Images were taken of plates in regions where there were monolayer of cells. The difference in average pixel intensities of individual cells and regions containing no cells to represent background were obtained. Image analyses were performed using Smart Capture VP software.

Dot Blot. CHO AA8 cells transfected with short interfering molecules at time points 0, 6, 12, 18 and 24 hours post transfection were lifted by washing three times with 1×PBS and incubating with trypsin for 5 minutes. Cells were washed with 1×PBS and resuspended in 1×PBS. Cell suspensions (approximately 10⁵ cells) were boiled for 10 minutes at 95° C. to obtain cellular lysates. Hybridization procedures were performed as described in Gibco's Blugene Nonradioactive Nucleic Acid Detection System to detect the presence of the short interfering molecules in the lysates. Probes used were antisense G6PD DNA sequence that had been biotinylated.

Statistics. The values presented in the CHO AA8 siRNA and siDNA time experiments represent the averages of five replicate experiments, the hybrid data represents the averages of three replicate experiments. The values presented of the hamster-human comparison time experiment represents the averages of two replicate experiments. Relative values were obtained by representing the average value of the positive control conditions as 100% and dividing the averages for the experimental conditions by the average positive control value. The error bars represent the standard deviation.

Experimental Results

Transfection of short interfering molecules using cationic liposomes inconsistently causes toxicity of cells and yields low transfection efficiency. CHO AA8 and Human MCF-7 cells were transfected with the short interfering molecules using cationic liposomes. Vital counts showed greater than 50% of the cells exposed to the transfection complexes died, regardless of the transfection reagent used. Five different cationic liposome transfection reagents were tried in order to minimize the toxicity and mortality of the cells, with Lipofectamine (Life Technologies) producing the lowest level of cell death. Only cells that looked healthy after transfection were assayed for G6PD activity.

Approximately 40-50% of cells transfected in a 35 mm plate appeared to be transfected with the short interfering molecules, based on cell color when assayed for G6PD enzymatic activity. Transfected and untransfected cells in monolayer cultures tended to occur in discrete patches, as indicated by the color of the cells. Only cells in the transfected regions were analyzed for gene silencing. In control plates where G6PD activity was not inhibited these regions of different intensities of color of the cells were not present, indicating that the G6PD assay was not producing the effect.

A colorimetric assay provided an efficient method to detect the presence of G6PD gene silencing in individual cells. The G6PD gene proved to be an advantageous choice to investigate siRNA-mediated gene silencing. To separate the efficiency of the transfection from the study of siRNA, it was important to be able to assay individual cells rather than obtain a population average. To do this, the enzymatic activity of the G6PD protein was assayed using a colorimetric assay developed by Stamato et al (22). Previous work using siRNA to silence genes in cultured mammalian cells by Elbashir et al (20) also used colorimetric techniques of fluorescent staining and luciferase activity to assay results. After transfection cells were incubated with a tetrazolium-based histochemical stain that contained glucose-6-phosphate (G6P) and nicotinamide dinucleotide triphosphate (NADP). The addition of G6P to cells activated G6PD gene transcription and protein synthesis. The enzymatic activities of G6PD coupled the oxidation of G6P and the reduction of NADP to NADPH, to create a cellular color change from white to purple. If the addition of siRNA with a sequence homologous to the G6PD gene sequence induced post-transcriptional gene silencing in CHO AA8 cells, then an insufficient amount of G6PD protein would be synthesized, resulting in a lack of G6PD enzymatic activity and inhibition of the color change reaction.

A reduction in inducible G6PD enzymatic activity exists in Chinese Hamster cells exposed to siRNA molecules. Relative changes of G6PD activity in siRNA-transfected cells were measured by comparing the color intensity of the cells to non-transfected cells that were also incubated with the histochemical stain. To ensure that the post-transcriptional gene silencing was a specific effect of the siRNA enzymatic activity was also measured in CHO AA8 cells transfected with a non-homologous nucleotide sequence, T7 primer, as well as cells that were exposed to cationic liposomes with no vector. CHO AA8 cells incubated with the histochemical stain in the absence of G6P served as a negative control for the assay. Images of cells were obtained after incubation and the pixel intensities based on the color of individual cells were measured to determine relative changes in G6PD activity.

G6PD activity could be detected in mammalian cells through the coupling of the oxidation of glucose-6-phosphate and the reduction of NADP by G6PD with a tetrazolium based histochemical stain.

Kinetics of siRNA induced gene silencing of G6PD. To determine the kinetics of siRNA post-transcriptional gene silencing of G6PD the colorimetric assay was performed at specific time points over the span of 96 hours after a 5 hour transfection to measure the presence of G6PD enzymatic activity. siRNA mediated gene silencing provided approximately a 60% reduction in G6PD activity for the first 24 hours post transfection. The cells began to regain expression of the G6PD gene at 48 hours and exhibited full expression by 96 hours after transfection.

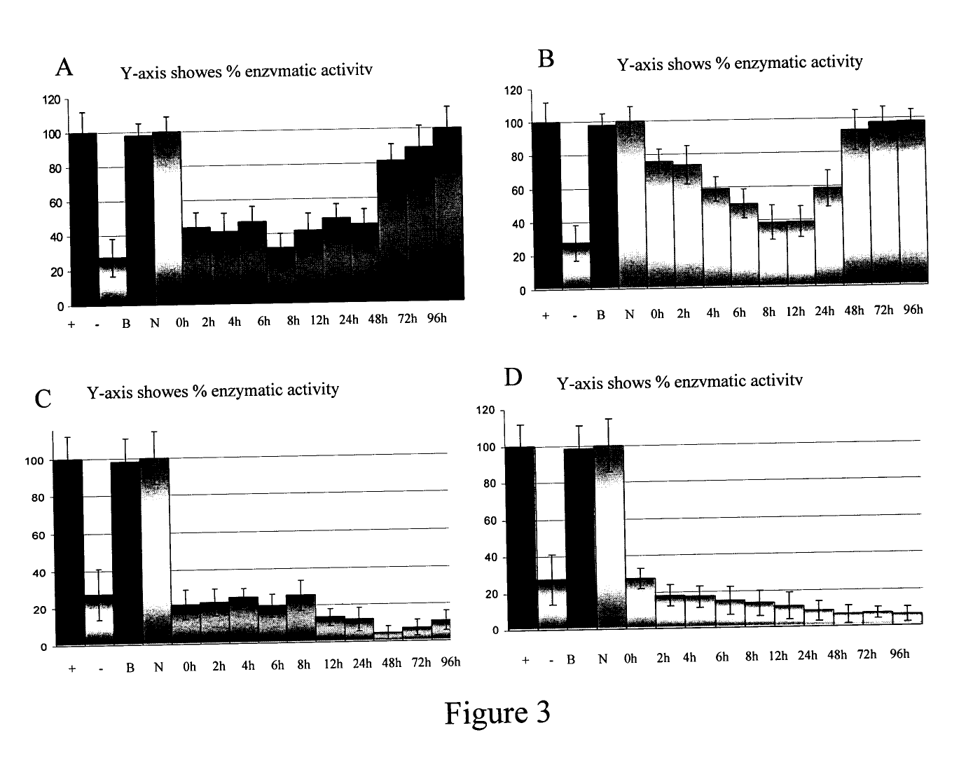

Referring to FIG. 3, (A) CHO AA8 cells transfected with siRNA molecules (B) Cells exposed to siDNA molecules (C) Introduction of short interfering hybrid molecules DNAs:RNAa and (D) RNAs:DNAa. Control reactions consisted of transfecting with si molecules (either RNA:RNA, RNA:DNA or DNA:DNA) that had the sequence of the T7 phage promoter primer (T), or exposure to cationic liposome complexes with no vector (B). All cells exposed to control tests exhibited 100% gene expression and enzymatic activity. Cells transfected with siDNA molecules exhibited the lowest degree of gene silencing effects while siRNA molecules provided a greater inhibition of gene expression. The length of silencing lasted approximately 24 hours for cells transfected with siRNA or siDNA molecules. Short interfering hybrid molecules of both DNAs:RNAa and RNAs:DNAa conformations exhibited the greatest degree and persistence of inhibition of endogenous gene expression. Effects continued to persist through 96 hours. Graphs A and B represent data from five replicate experiments and data from graphs C and D represent data from three replicate experiments.

Cells exhibit a differential response in G6PD gene silencing when exposed to short interfering molecules of different nucleic acid composition. Because the mechanism of RNAi mediated by siRNA is not clear it was questioned whether post-transcriptional gene silencing was a specific effect of short interfering sequences made of RNA or could siRNA molecules with variations in their nucleic acid composition provide gene silencing effects. To test this, siDNA sequences and short interfering hybrid molecules composed of both RNA and DNA, identical in sequence to the siRNA vectors used were transfected into CHO AA8 cells and G6PD enzymatic activity was assayed again at designated time points over the span of 96 hours post transfection. Two different hybrid molecules were constructed that differed in which nucleic acid the sense and antisense strands were composed of. Analysis of the cells suggested that a differential response of G6PD silencing existed among the different short interfering molecules used. Cells transfected with siDNA molecules showed the lowest degree of gene silencing and maximum inhibition of expression was not seen until 12 hours post transfection. In contrast cells transfected with siRNA molecules showed a decrease in expression as early as 0 hours after transfection with a greater degree of silencing compared to that provided by the siDNA molecules. Both effects of siDNA and siRNA molecules lasted for approximately 24 hours and normal expression levels were reached by 96 hours.

CHO AA8 cells transfected with the short interfering hybrid molecules of both DNAs:RNAa and RNAs:DNAa exhibited the greatest decrease in G6PD enzymatic activity with the greatest persistence. Cells transfected with DNAs:RNAa showed a decrease in G6PD as early as 0 hours after transfection with percent relative activity at approximately 20%. These effects persisted throughout the time course of the experiment with amount of activity remaining at approximately 20% or lower. Similar effects were seen with cells transfected with RNAs:DNAa molecules. Referring to FIG. 3, percent enzymatic activity remained at or below approximately 20% throughout the experiment. A dot blot was performed to detect the presence of the short interfering hybrid molecules. Nothing was detected, which demonstrates only that the intracellular concentration of the molecules was too low to be detected.

The presence of G6PD activity was assayed for in cells exposed to the hybrid molecules every 24 hours between 120-192 hours post transfection to determine how long the, effects last with the hybrid constructs. The presence of G6PD activity increased to about 40% by 120 hours but remained at this level through 192 hours. A dot blot was performed to detect the presence of the short interfering hybrid molecules. Nothing was detected, which demonstrates only that the intracellular concentration of the molecules was too low to be detected.

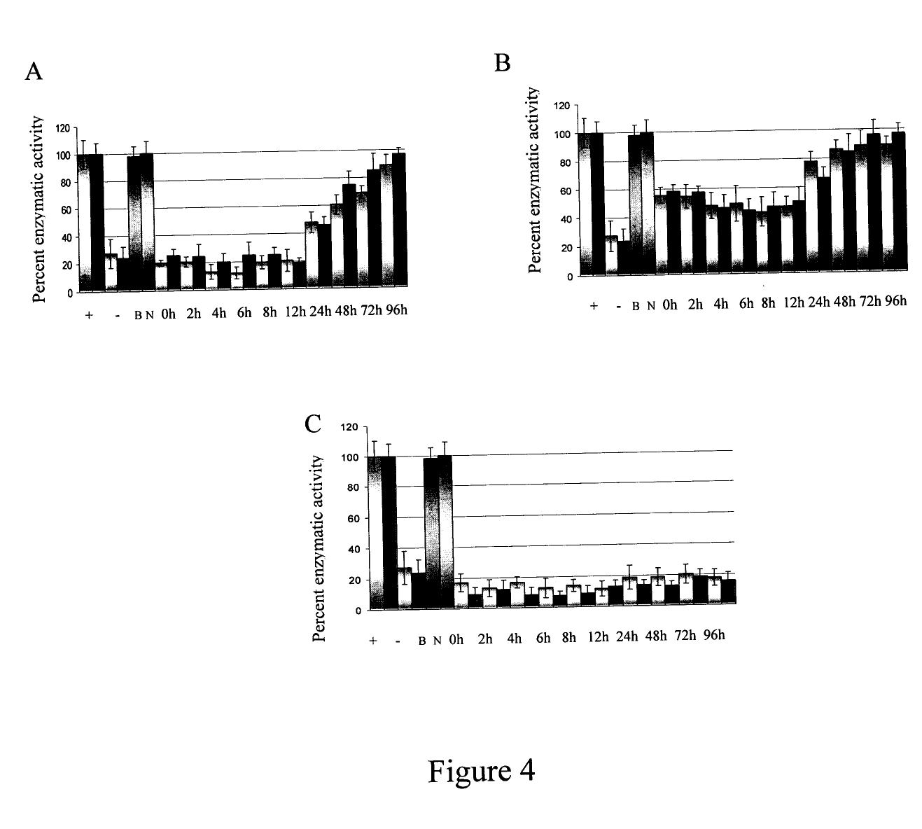

A differential response in siRNA-mediated gene silencing with varied nucleic acid composition possibly exists in all mammalian cells. To show that the differential response was not a specific effect of hamster cells a comparison time course study of the persistence of short interfering molecules with variations in their nucleic acid composition was done in human and hamster cells. This experiment also addressed the effects of varying the sequence of the gene the short interfering molecule is homologous to. The molecules were identical to a sequence in both the hamster and human G6PD coding region. FIG. 4 shows that a differential response was also present in the Human MCF-7 cells suggesting the possible universality of this application to all cultured mammalian cells. Cells transfected with siDNA molecules exhibited the lowest degree of gene silencing while siRNA molecules provided a greater degree of inhibition of gene expression. The silencing effects of both siRNA and siDNA showed a loss by approximately 24 hours post transfection with full expression regained by 96 hours. The hybrid molecules in both human and hamster cells offered the greatest reduction in gene silencing with long term inhibition of endogenous gene expression. Only hybrid molecules composed of a RNA sense strand and a DNA antisense strand were used due to the similarity of the results obtained for both hybrid molecules in the previous experiment involving hamster cells only.

Referring to Figure CHO AA8 and Human MCF-7 cells transfected with (A) siRNA molecules (B) siDNA molecules and (C) short interfering hybrid molecule of RNAs:DNAa composition. As the sequence used here is a different sequence then that used in the first series of experiments in the CHO AA8 cells, these results demonstrate both that the differential response was not a cell-specific effect, nor was it a sequence-specific effect. The introduction of siDNA molecules resulted in the lowest inhibition of gene expression, while siRNA molecules provided a greater degree of gene silencing. Both effects in both cell lines lasted for approximately 24 hours post transfection. The short interfering hybrid molecule exhibited the greatest degree and persistence of inhibition of G6PD gene expression that lasted for the time course of the experiment. Data from all three graphs represent data from two replicate experiments.

In addition to showing the potential use this application has in mammalian cells, these experiments demonstrate that a differential response is present regardless of the sequence of the coding region to which the short interfering molecules are homologous. The initial experiments testing the effects of nucleic acid composition in hamster cells utilized a different short interfering sequence than the human-hamster comparison experiment, and both sequences were homologous to undistinguished regions of the coding strand. Yet both resulted in gene silencing with a differential response and a long-term inhibition provided by the hybrid molecules.

Bacterial Cells

Only unaided delivery of siHybrids was used. Unaided delivery involved adding the siHybrids directly to the media of the bacteria cultures.

1. Effective dose of cat siHybrids: UltraMAX DH5α−FT Competent E.coli cells (Invitrogen, Carlsbad, Calif.) were transformed by heat shock with the pBC SK+plasmid (Promega, Madison, Wis.) encoding the chloramphenicol acetyl tranferase resistant protein (cat gene). The transformants were cultured in 2 mls of Luria-Bertani (LB) media containing 25 μg/ml of chloramphenicol antibiotic (Sigma, St. Louis, Mo.) in the presence of the cat siHybrid constructs. To determine the effective dose of the cat siHybrids, cells were grown in the presence of 0.125, 0.25, .50, 1.0, 2.0, 4.0, and 8.0 μg/ml of the constructs. siHybrids were 21 base pair in length, homologous to a specific sequence within the coding region of the cat gene of the pBC SK+plasmid. Constructs were designed according to Tuschl et al guidelines. (See “The siRNA user guide” at the web address mpibpc.gwdg.de/abteilungen/100/105/sirna.html) The sense strand was composed of RNA of the sequence 5′CGGUGGUAUAUCCAGUGAUUUUU3′ (Dharmacon, Lafayette, CO). The antisense strand was composed of DNA of the sequence 5′ AAATCACTGGATATACCACCGTT 3′ (Sigma Genosys, The Woodlands, Tex.). Control conditions included a positive control, which contained cells grown in LB-chloramphenicol media, cultures grown in LB-chloramphenicol media in the presence of 4 μg/ml siRNA that is identical in sequence to the cat siHyrbid. To show that the action of the siHybrid was sequence specific, a siHybrid construct that was not homologous (NH) to any region of the E.coli genome was also used. The sense strand of the non-homologous construct was composed of RNA of the sequence 5′ CUGGCCAGCCACAUAGGAGUUUU 3′ (Dharmacon). The antisense strand was composed of DNA of the sequence 5′ AACTCCTATGTGGCTGGCCAGTT 3′ (Sigma Genosys). Cultures were incubated on a shaker at 250 rpm at 37° C. overnight. Following incubation, cultures were diluted to 1:1000 in LB media and 10 μl of dilutant were plated on LB/agar plates supplemented with 25 μg/ml of chloramphenicol antibiotic. To obtain uniform colonies on the plates, soda lime glass beads, 5 mm in diameter (VWR, Westchester, Pa.) were applied to the plates and shaken by hand. Plates were incubated upside down at 37° C. overnight. To quantify the occurrence of gene silencing of gene silencing activity by the siHybrids, colony forming units (CFU) were calculated using the following equation: [(number of colonies/10 ml plated)×(dilution factor)×(2000 μl)].

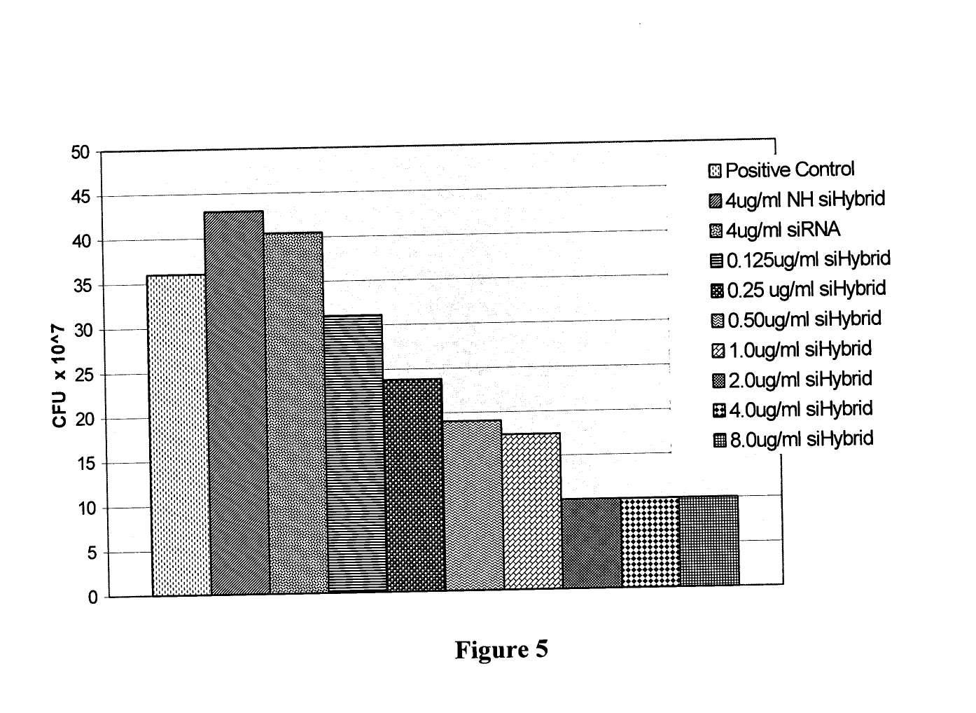

Referring to FIG. 5, cat siHybrid activity was measured and was expressed in 10⁷ colony forming units. The control, non-homologous siHybrid, and siRNA bars all exhibited similar growth in the area of 36.0-43.0×10⁷ CFU The results indicate that colony growth is inversely proportional to cat siHybrid concentration. The cat siHybrid concentrations at 2.0 μg/ml, 4.0 μg/ml and 8.0 μg/ml all showed a 72.2% decrease in colony formation compared to the control. There was an average decrease of 23.6% in colony growth for every dose of cat siHybrid added. The percent error was calculated to be about 5% for all experimental techniques here.

2. Silencing of the folA gene using siHybrids. Cultures of MG1655 E.coli cells were initiated in 2 mls of M9 minimal media in the presence of 4 μg/ml siHybrids homologous to a sequence of the coding region of the folA gene, whose protein product was dihydrofolate reductase. The sense strand was composed of RNA of the sequence 5′ UCUCGCCUGGUUUAAACGCAAUU 3′ (Johns Hopkins Synthesis Facility, Baltimore, Md.). The antisense strand was composed of DNA of the sequence 5′ TTGCGTTTAAACCAGGCGAGATT 3′ (Johns Hopkins Synthesis Facility). Silencing of the folA gene prevents growth of E.coli cells in minimal media due to their inability to synthesize purines and pyrimidines. There were six experimental conditions, which included the positive control containing cells in minimal media, cells in minimal media with 4 μg/ml of the folA siHybrid construct, cells in minimal media with 4 μg/ml of the folA siRNA (identical in sequence to the folA siHybrid) construct, cells in minimal media with 4 μg/ml of a non-homologous siHybrid construct, and cells grown in minimal media with 4 μg/ml folA siHybrid and supplemented with 20 mM thymidine, cytidine, adenosine and guanosine (Sigma). Cells were also grown in minimal media supplemented with 50 μg/ml trimethoprim (Sigma), which is an antibiotic that specifically inhibits the action of dihydrofolate reductase, which served as a pharmacological comparison. Cultures were grown over night at 37° C. and shaken at 250 rpm. Cultures were diluted to 1:1000 and 1:100,000 and plated out the next day. Colonies were counted and CFU were calculated for each experimental condition.

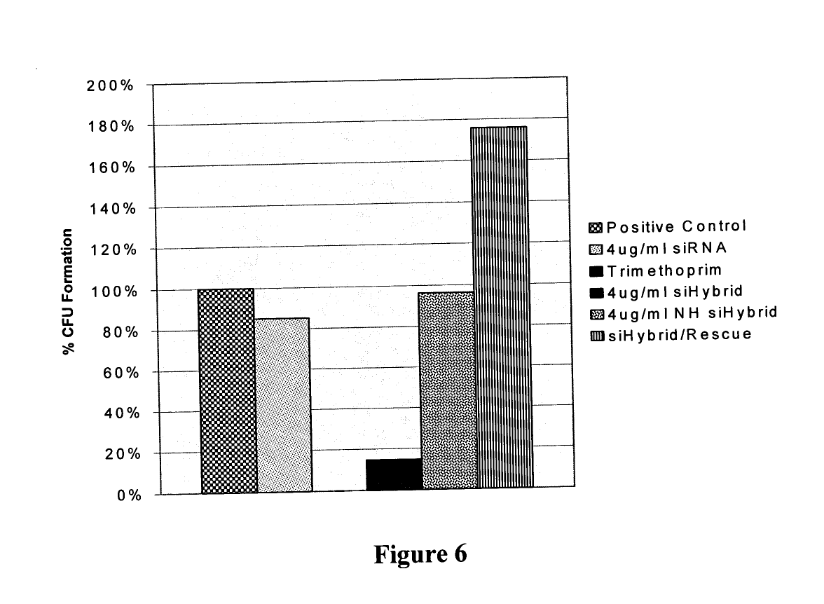

Referring to FIG. 6, the CFU value obtained for the positive control (cells grown in minimal media alone) was set at 100% and all other conditions were expressed relative to that value. There was a significant reduction of over 80% in CFU formation for cultures grown in the presence of the folA siHybrids. There was an insignificant difference between the amount of CFU formed in the positive control, cultures grown in the presence of the folA siRNA, and cultures grown in the presence of the non-homologous siHybrid. The cultures grown in the presence of the folA siHybrids as well as the purines and pyrimidines (siHybrid/Rescue) exhibited over 100% CFU formation. It has been experimentally determined (data not shown) that this is caused by the growth rates of the rescue cultures being significantly faster than that of the positive control. It is hypothesized that the growth rate is faster due to the fact that the cells in the rescue cultures did not need to synthesize their own purines and pyrimidines, so division could occur at a faster rate.

All publications and patents mentioned in the above specification are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

The invention claimed is:

1. A composition comprising:
an siHybrid comprising (1) a hybridized complimentary portion, and (2) at least one over-hanging 3′ end portion, said siHybrid having a first single strand sequence of nucleic acid or nucleic acid analog hybridized to a second single strand sequence of nucleic acid or nucleic acid analog, wherein the second single strand is of a different type of nucleic acid or nucleic acid analog than the first single strand sequence.

2. The composition of claim 1, wherein the first single strand sequence and the second single strand sequence are selected from the group consisting of DNA, RNA and PNA.

3. The composition of claim 1, wherein the hybridized complimentary portion of said composition has a length of ten to one hundred base pairs.

4. The composition of claim 1, wherein said over-hanging 3′ end is 2-3 bases in length.

5. The composition of claim 1, wherein the hybridized complimentary portion of the composition has a length of 19-21 base pairs.

6. The composition of claim 1, wherein said siHybrid has two over-hanging 3′ ends.

7. The composition of claim 6, wherein~said two over-hanging 3′ ends are each 2 bases in length and the hybridized portion of the composition has a length of 21 base pairs.

8. A composition comprising:
an siHybrid comprising (1) a hybridized complimentary portion having a length of 19-21 base pairs, and (2) two over-hanging 3′ ends each 2-3 bases in length, wherein said siHybrid has a first single strand sequence of nucleic acid or nucleic acid analog hybridized to a second single strand sequence of nucleic acid or nucleic acid analog, wherein the second single strand is of a different type of nucleic acid or nucleic acid analog than the first single strand sequence.

9. The composition of claim 8, wherein the first single strand sequence and the second single strand sequence are selected from the group consisting of DNA, RNA and PNA.

10. A composition comprising:
an siHybrid comprising (1) a hybridized complimentary portion having a length of 21 base pairs, and (2) two over-hanging 3′ ends each 2 bases in length, wherein said siHybrid has a first single strand sequence of nucleic acid or nucleic acid analog hybridized to a second single strand sequence of nucleic acid or nucleic acid analog, wherein the second single strand is of a different type of nucleic acid or nucleic acid analog than the first single strand sequence.

11. The composition of claim 10, wherein the first single strand sequence and the second single strand sequence are selected from the group consisting of DNA, RNA and PNA.

12. A method comprising:
providing a first single strand sequence of nucleic acid or nucleic acid analog;
providing a second single strand sequence of nucleic acid or nucleic acid analog that is of a different type of nucleic acid or nucleic acid analog than said first single strand sequence; and
hybridizing said first single strand sequence and said second single strand sequence to make an siHybrid having (1) a hybridized complimentary portion, and (2) at least one 3′ over-hanging end.

13. The method of claim 12, wherein the first single strand sequence and the second single strand sequence are selected from the group consisting of DNA, RNA and PNA.

14. The method of claim 12, wherein the hybridized portion of the siHybrid has a length of ten to one hundred base pairs.

15. The method of claim 12, wherein said over-hanging 3′ end is 2-3 bases in length.

16. The method of claim 12, wherein the hybridized complementary portion of the siHybrid has a length of 19-21 base pairs.

17. The method of claim 12, wherein said siHybrid has two over-hanging 3′ ends each 2 bases in length and said hybridized portion of the siHybrid has a length of 21 base pairs.

18. The method of claim 12, further comprising:
contacting said siHybrid directly to a substrate or to a substrate using a transfection agent to silence at least one gene.

19. The method of claim 18, wherein the hybridized portion of the siHybrid has a length of ten to one hundred base pairs.

20. The method of claim 18, wherein said over-hanging 3′ end is 2-3 bases in length.

21. The method of claim 18, wherein the hybridized complementary portion of the siHybrid has a length of 19-21 base pairs.

22. The method of claim 18, wherein said si Hybrid has two over-hanging 3′ ends each 2 bases in length and said hybridized portion of the siHybrid has a length of 21 base pairs.

23. The method of claim 18, wherein said substrate is an organism or a cell.

24. The method of claim 23, wherein said organism is a virus, a prokaryote, a bacterium, a eukaryote, eukaryotic cells, a vertebrate, a mammal, a primate, a human, human cells, a plant, an insect, or a fungus.

25. A method comprising:
providing an siHybrid having a first single strand sequence of nucleic acid or nucleic acid analog hybridized to a second single strand sequence of nucleic acid or nucleic acid analog that is of a different type of nucleic acid or nucleic acid analog than said first single strand sequence, wherein said siHybrid has (1) a hybridized complimentary portion, and (2) at least one over-hanging 3′ end; and
contacting said siHybrid directly to a substrate or to a substrate using a transfecting agent to silence at least one gene.

26. The method of claim 25, wherein the first single strand sequence and the second single strand sequence are selected from the group consisting of DNA, RNA and PNA.

27. The method of claim 25, wherein the hybridized portion of the siHybrid has a length of ten to one hundred base pairs.

28. The method of claim 25, wherein said over-hanging 3′ end is 2-3 bases in length.

29. The method of claim 25, wherein the hybridized complementary portion of the siHybrid has a length of 19-21 base pairs.

30. The method of claim 25, wherein said siHybrid has two over-hanging 3′ ends each 2 bases in length and said hybridized portion of the siHybrid has a length of 21 base pairs.

31. The method of claim 25, wherein said substrate is an organism or a cell.

32. The method of claim 31, wherein said organism is a virus, a prokaryote, a bacterium, a eukaryote, eukaryotic cells, a vertebrate, a mammal, a primate, a human, human cells, a plant, an insect, or a fungus.

33. A method comprising:
providing a first single strand sequence of nucleic acid or nucleic acid analog, said sequence common to a plurality of genes;
providing a second single strand sequence of nucleic acid or nucleic acid analog that is of a different type of nucleic acid or nucleic acid analog than said first single strand sequence; and
hybridizing said first single strand sequence with said second single strand sequence to make an siHybrid having (1) a hybridized complimentary portion and (2) at least one 3′ over-hanging end.

34. The method of claim 33, wherein the first single strand sequence and the second single strand sequence are selected from the group consisting of DNA, RNA and PNA.

35. The method of claim 33, further comprising:
contacting said siHybrid directly to a substrate or to a substrate using a transfecting agent to silence said plurality of genes.

36. The method of claim 33, wherein the hybridized complimentary portion of the siHybrid has a length of ten to one hundred base pairs.

37. The method of claim 33, wherein said 3′ over-hanging end is 2-3 bases in length and said hybridized complimentary portion is 19-21 base pairs in length.

38. The method of claim 33, wherein said siHybrid has two 3′ over-hangs each 2 bases in length and said hybridized portion of the siHybrid has a length of 21 base pairs.

39. The method of claim 35, wherein the substrate is an organism or a cell

40. The method of claim 39, wherein said organism is a virus, a prokaryote, a bacterium, a eukaryote, eukaryotic cells, a vertebrate, a mammal, a primate, a human, human cells, a plant, an insect, or a fungus.

41. A method comprising:
providing a plurality of siHybrids each with a different sequence, said siHybrids each comprising a first single strand sequence of nucleic acid or nucleic acid analog hybridized to a second single strand sequence of nucleic acid or nucleic acid analog that is a different type of nucleic acid or nucleic acid analog from said first single strand, wherein each siHybrid has (1) a hybridized complimentary portion, and (2) at least one 3′ over-hanging end; and
contacting said plurality of siHybrids directly to a substrate or to a substrate using a transfecting agent to silence at least one gene.

42. The method of claim 41, wherein the first single strand sequence and the second single strand sequence are selected from the group consisting of DNA, RNA and PNA.

43. The method of claim 41, wherein the hybridized portion of the siHybrid has a length of ten to one hundred base pairs.

44. The method of claim 41, wherein said over-hanging 3′ end is 2-3 bases in length.

45. The method of claim 41, wherein the hybridized complementary portion of the siHybrid has a length of 19-21 base pairs.

46. The method of claim 41, wherein said siHybrid has two over-hanging 3′ ends each 2 bases in length and said hybridized portion of the siHybrid has a length of 21 base pairs.

47. The method of claim 41, wherein the substrate is an organism or a cell.

48. The method of claim 47, wherein said organism is a virus, a prokaryote, a bacterium, a eukaryote, eukaryotic cells, a vertebrate, a mammal, a primate, a human, human cells, a plant, an insect, or a fungus.

49. A method comprising:
providing a first plurality of single strand sequences of nucleic acid or nucleic acid analog each with a different sequence;
providing a second plurality of single strand sequences of nucleic acid or nucleic acid analog, wherein said second plurality of single strands are different types of nucleic acid or nucleic acid analog from said first plurality of single strands; and
hybridizing said first plurality of single strands with said second plurality of single strands to make a plurality of siHybrids wherein each siHybrid has (1) a hybridized complimentary portion, and (2) at least one 3′ over-hanging end portion.

50. The method of claim 49, further comprising:
contacting said plurality of siHybrids directly to a substrate or to a substrate using a transfecting agent to silence at least one gene.

51. The method of claim 49, wherein the hybridized complimentary portions of said siHybrid have a length of ten to one hundred base pairs.

52. The method of claim 49, wherein said 3′ over-hangs are 2-3 bases in length.

53. The method of claim 49, wherein said hybridized complimentary portions are 19-21 base pairs in length.

54. The method of claim 49, wherein each siHybrid has two 3′ over-hangs each 2 bases in length and the hybridized portion of said plurality of siHybrid are 21 base pairs in length.

55. The method of claim 50, wherein the substrate is an organism or a cell.

56. The method of claim 55, wherein said organism is a virus, a prokaryote, a bacterium, a eukaryote, eukaryotic cells, a vertebrate, a mammal, a primate, a human, human cells, a plant, an insect, or a fungus.

57. A method comprising:
providing a single strand of DNA, having 23 bases with a sequence of first 21 bases from 5′ complimentary to a targeted gene;
providing a single strand RNA, having 23 bases with a sequence of first 21 bases from 5′ complimentary to said sequence of first 21 bases from 5′ of said single strand DNA;
hybridizing said single strand DNA to said single strand RNA to form an siHybrid having (1) a 21 base pair hybridized portion and (2) a two base over-hanging portion at each 3′ end.

58. The method of claim 57, further comprising:
contacting said siHybrid to a cell or an organism, said organism selected from the group consisting of a virus, a prokaryote, a bacterium, a eukaryote, eukaryotic cells, a vertebrate, a mammal, a primate, a human, human cells, a plant, an insect, and a fungus.

59. A method comprising:
providing a single strand of DNA, having 23 bases with a sequence of first 21 bases from 5′ complimentary to a targeted gene hybridized to a single strand RNA, having 23 bases with a sequence of first 21 bases from 5′ complimentary to said sequence of first 21 bases from 5′ of said single strand DNA to form an siHybrid having (1) a 21 base pair hybridized portion and (2) a two base over-hanging portion at each 3′ end; and
contacting said siHybrid to a substrate.

60. The method of claim 59, wherein said substrate is a cell or an organism, said organism selected from the group consisting of a virus, a prokaryote, a bacterium, a eukaryote, eukaryotic cells, a vertebrate, a mammal, a primate, a human, human cells, a plant, an insect, and a fungus.

61. A method comprising:
providing a single strand of RNA, having 23 bases with a sequence of first 21 bases from 5′ complimentary to a targeted gene;
providing a single strand DNA, having 23 bases with a sequence of first 21 bases from 5′ complimentary to said sequence of first 21 bases from 5′ of said single strand RNA;
hybridizing said single strand RNA to said single strand DNA to form an siHybrid having (1) a 21 base pair hybridized portion and (2) a two base over-hanging portion at each 3′ end.

62. The method of claim 61, further comprising:
contacting said siHybrid to a cell or an organism, said organism selected from the group consisting of a virus, a prokaryote, a bacterium, a eukaryote, eukaryotic cells, a vertebrate, a mammal, a primate, a human, human cells, a plant, an insect, and a fungus.

63. A method comprising:
providing a single strand of RNA, having 23 bases with a sequence of first 21 bases from 5′ complimentary to a targeted gene hybridized to a single strand DNA, having 23 bases with a sequence of first 21 bases from 5′ complimentary to said sequence of first 21 bases from 5′ of said single strand RNA to form an siHybrid having (1) a 21 base pair hybridized portion and (2) a two base over-hanging portion at each 3′ end; and
contacting said siHybrid to a substrate.

64. The method of claim 63, wherein said substrate is a cell or an organism, said organism selected from the group consisting of a virus, a prokaryote, a bacterium, a eukaryote, eukaryotic cells, a vertebrate, a mammal, a primate, a human, human cells, a plant, an insect, and a fungus.
